(19) United States
(12) Patent Application Publication
(10) Pub. No.: US 2015/0299796 A1
(43) Pub. Date: Oct. 22, 2015

(54) PREDICTION OF TREATMENT RESPONSE TO JAK/STAT INHIBITOR

(71) Applicants: Alexander CAO, Cambridge, MA (US); Michael MORRISSEY, Cambridge, MA (US); Dmitriy SONKIN, Cambridge, MA (US); Michael PALMER, Cambridge, MA (US)

(72) Inventors: Alexander CAO, Cambridge, MA (US); Michael MORRISSEY, Cambridge, MA (US); Dmitriy SONKIN, Cambridge, MA (US); Michael PALMER, Cambridge, MA (US)

(21) Appl. No.: 14/417,632

(22) PCT Filed: Jul. 24, 2013
(86) PCT No.: PCT/US2013/051824

§ 371 Date: Jan. 27, 2015

Related U.S. Application Data
(60) Provisional application No. 61/676,484, filed on Jul. 27, 2012.
(60) Provisional application No. 61/769,271, filed on Feb. 26, 2013.
(60) Provisional application No. 61/829,327, filed on May 31, 2013.

(51) Int. Cl. C12Q 1/68; A61K 31/519

(52) U.S. Cl. CPC C12Q 1/6886; A61K 31/519; C12Q 2600/158; C12Q 2600/112; C12Q 2600/16

(57) ABSTRACT

The invention includes, in part, a method of selecting a subject having cancer for treatment with a JAK/STAT inhibitor and a method of determining if a therapeutically effective dose of a JAK/STAT inhibitor has been administered.

### FIELD OF THE INVENTION

The present invention relates to a method of treatment of cancer.

### BACKGROUND OF THE INVENTION

The JAK-STAT pathway is one of the important signaling pathways downstream of cytokine receptors. Following binding of a ligand to its receptor, receptor-associated JAKs are activated. STAT proteins, upon phosphorylation by JAKs, dimerize and translocate to the nucleus. Inside the nucleus, the activated STAT proteins modulate the expression of target genes (Imada et al. Molecular Immunology 2000, 37: 1-11).

The JAK family consists of four non-receptor protein tyrosine kinases, JAK1, JAK2, JAK3, and TYK2 (Stark et al., Immunology 36: 503-514). JAK1, JAK2, and TYK2 are expressed ubiquitously in mammals, while JAK3 is expressed mainly in hematopoietic cells. Once activated by cytokines or growth factors, JAKs serve as docking sites for STATs. A number of STAT molecules, including STAT 1, 3, 4, 5 and 6, have been identified (Murray P J 2007 J Immunology 178:2623-29; Rawlings J S et al., 2004 J Cell Sci. 117:1281). Activated STATs translocate from the cytoplasm to the nucleus where they modulate the transcription rate of target genes (Rawlings J S et al., 2004 J Cell Sci. 117:1281; Stark et al., 2012, Immunology 36: 503-514).

JAK-STAT signaling has been implicated in multiple human pathogenesis. The genetic aberration of JAK2 and the associated activation of STAT in myeloproliferative neoplasms (MPN) is one example of the involvement of this pathway in human neoplasia. Additionally, activated JAK-STAT has been suggested as a survival mechanism for human cancers.

Given the importance of JAK-STAT activation in human diseases, it becomes important to identify patients with activated JAK-STAT pathways. The detection of JAK activation through the measurement of phospho-JAK in clinical samples is subject to many technical and logistical variables.

### SUMMARY OF THE INVENTION

The present invention is based on the finding that particular biomarkers can be used to select individuals who have an activated STAT pathway. Specifically, it was found that an increased level of mRNA expression of one or more biomarkers listed in Table 1, e.g., the mRNA expression of a biomarker listed in Table 1 in a sample from an individual having cancer compared to a control, can be used to predict whether that individual has an activated STAT pathway.

In one aspect, the invention includes a method of selecting a subject having a hematological malignancy for treatment with a STAT signaling inhibitor such as a JAK/STAT inhibitor. The method includes determining the level of expression of at least one, two, three, four, five, six, or more biomarkers listed in Table 1 in a biological sample derived from the subject, thereby to predict an increased likelihood of response to a STAT signaling inhibitor, e.g., a JAK/STAT inhibitor. In one embodiment, invention includes determining the level of expression of two biomarkers from Table 1 such as PIM1 and CISH. In another embodiment, the invention includes determining the expression of four biomarkers from Table 1 such as PIM1, CISH, SOCS2, and ID1. In another embodiment, the invention includes determining the level of expression of six biomarkers in Table 1. The at least six biomarkers can include PIM1, CISH, SOCS2, ID1, LCN2, and EPOR. In another embodiment, the invention includes determining the level of expression of at least seven biomarkers in Table 1. The at least seven biomarkers can include PIM1, CISH, SOCS2, ID1, LCN2, EPOR and EGR1. The mRNA expression can be determined using any method known in the art. In particular mRNA expression of the biomarkers of Table 1 can be determined using reverse Transcriptase PCR (RT-PCR).

In one embodiment, the JAK/STAT inhibitor is a JAK2 inhibitor such as (R)-3-cyclopentyl-3-[4-(7H-pyrrolo[2,3-d]pyrimidin-4-yl)-1H-pyrazol-1-yl]propanenitrile; or a pharmaceutically acceptable salt thereof.

In one embodiment, the hematological malignancy is leukemia, lymphoma or myeloma.

In another aspect, the invention includes a kit comprising a plurality of agents for determining the level of mRNA expression of four or more biomarkers listed in Table 1 in a sample and instructions for use.

In another aspect, the invention includes a method of selecting a subject having a hematological malignancy for treatment with a STAT signaling inhibitor such as a JAK/STAT inhibitor, the method includes determining an increase in the level of mRNA expression of at least one or more biomarkers listed in Table 1 in a biological sample derived from the subject; wherein an increase in the level of mRNA expression of one or more biomarkers in Table 1 is indicative that the patient is more likely to respond to treatment with a STAT signaling inhibitor such as a JAK/STAT inhibitor; and administering a STAT signaling inhibitor such as a JAK/STAT inhibitor to the patient who has an increased level of mRNA expression of one or more biomarkers in Table 1. The JAK/STAT inhibitor can be any JAK2 inhibitor such as (R)-3-cyclopentyl-3-[4-(7H-pyrrolo[2,3-d]pyrimidin-4-yl)-1H-pyrazol-1-yl]propanenitrile; or a pharmaceutically acceptable salt thereof.

In another aspect, the invention includes a method of selecting a subject having a hematological malignancy for treatment with a STAT signaling inhibitor, e.g., a JAK/STAT inhibitor, the method comprising administering a STAT signaling inhibitor, e.g., a JAK/STAT inhibitor to a selected patient, wherein a sample from the selected patient has been determined to have an increased level of mRNA expression of one or more biomarkers listed in Table 1.

In another aspect, the invention includes selecting a subject having a hematological malignancy for treatment with a STAT signaling inhibitor, e.g., a JAK/STAT inhibitor, the method comprising either
selectively administering a therapeutically effective amount of a STAT signaling inhibitor, e.g., a JAK/STAT inhibitor to a selected patient on the basis that the selected patient has been determined to have an increased level of mRNA expression of one or more biomarkers listed in Table 1; or
selectively administering a therapeutically effective amount of an inhibitor which is not a STAT signaling inhibitor, e.g., a JAK/STAT inhibitor to the selected subject on the basis that the sample does not have an increased level of mRNA expression of one or more biomarkers listed in Table 1.

In another aspect, the invention includes selecting a subject having a hematological malignancy for treatment with a STAT signaling inhibitor, e.g., a JAK/STAT inhibitor, the method comprising either
determining the level of expression of at least one or more biomarkers listed in Table 1 in a biological sample derived from the subject, and either
selectively administering a therapeutically effective amount of a STAT signaling inhibitor, e.g., a JAK/STAT inhibitor to a selected patient on the basis that the selected patient has been determined to have an increased level of mRNA expression of one or more biomarkers listed in Table 1; or
selectively administering a therapeutically effective amount of an inhibitor which is not a STAT signaling inhibitor to the selected subject on the basis that the sample does not have an increased level of mRNA expression of one or more biomarkers listed in Table 1.

In another aspect, the invention includes selecting a subject having a hematological malignancy for treatment with a STAT signaling inhibitor, e.g., a JAK/STAT inhibitor, the method comprising:
determining the level of expression of at least one or more biomarkers listed in Table 1 in a biological sample derived from the subject, and
thereafter selecting the subject for treatment with a therapeutically effective amount of a STAT signaling inhibitor, e.g., a JAK/STAT inhibitor on the basis that the selected patient has been determined to have an increased level of mRNA expression of one or more biomarkers listed in Table 1 and recording the result of the determining step on a tangible or intangible media form for use in transmission.

In another aspect, the invention includes a method for producing a transmittable form of information for predicting the responsiveness of a patient to a STAT signaling inhibitor, e.g., a JAK/STAT inhibitor, comprising:
a) determining an increased likelihood that the patient will respond to treatment with the STAT signaling inhibitor, e.g., a JAK/STAT inhibitor based on an increased level of expression of two or more biomarkers in Table 1; and
b) recording the result of the determining step on a tangible or intangible media form for use in transmission.

In another aspect, the invention includes a method of determining if a therapeutically effective dose of a STAT signaling inhibitor, e.g., a JAK/STAT inhibitor such as (R)-3-cyclopentyl-3-[4-(7H-pyrrolo[2,3-d]pyrimidin-4-yl)-1H-pyrazol-1-yl]propanenitrile; or a pharmaceutically acceptable salt thereof, is administered to a subject having a hematological malignancy comprising determining the level of mRNA expression of at least one or more biomarkers listed in Table 1 in a biological sample derived from the subject, wherein a decrease in mRNA expression following administration of (R)-3-cyclopentyl-3-[4-(7H-pyrrolo[2,3-d]pyrimidin-4-yl)-1H-pyrazol-1-yl]propanenitrile; or a pharmaceutically acceptable salt thereof, of at least one or more biomarkers listed in Table 1 in the biological sample is predictive that a therapeutic dose of the JAK/STAT inhibitor such as (R)-3-cyclopentyl-3-[4-(7H-pyrrolo[2,3-d]pyrimidin-4-yl)-1H-pyrazol-1-yl]propanenitrile; or a pharmaceutically acceptable salt thereof has been administered.

In still another aspect, the invention includes a STAT signaling inhibitor, e.g., a JAK/STAT inhibitor such as (R)-3-cyclopentyl-3-[4-(7H-pyrrolo[2,3-d]pyrimidin-4-yl)-1H-pyrazol-1-yl]propanenitrile; or a pharmaceutically acceptable salt thereof for use in treating a hematological malignancy, characterized in that a therapeutically effective amount of said compound or its pharmaceutically acceptable salt is administered to the patient on the basis of an increase in the level of expression of at least one or more biomarkers listed in Table 1 in a biological sample.

In still another aspect, the invention includes a JAK/STAT inhibitor such as (R)-3-cyclopentyl-3-[4-(7H-pyrrolo[2,3-d]pyrimidin-4-yl)-1H-pyrazol-1-yl]propanenitrile; or a pharmaceutically acceptable salt thereof for use in treating a hematological malignancy, characterized in that a therapeutically effective amount of said compound or its pharmaceutically acceptable salt is administered to the patient on the basis of the patient having an increase in the level of expression of at least four or more biomarkers listed in Table 1 in a biological sample.

In still another aspect, the invention includes a JAK/STAT inhibitor such as (R)-3-cyclopentyl-3-[4-(7H-pyrrolo[2,3-d]pyrimidin-4-yl)-1H-pyrazol-1-yl]propanenitrile; or a pharmaceutically acceptable salt thereof for use in treating a hematological malignancy, characterized in that a therapeutically effective amount of said compound or its pharmaceutically acceptable salt is administered to the patient on the basis of the patient having an increase in the level of expression of at least six or all of the biomarkers listed in Table 1 in a biological sample.

In still another aspect, the invention includes a STAT signaling inhibitor, e.g., a JAK/STAT inhibitor such as (R)-3-cyclopentyl-3-[4-(7H-pyrrolo[2,3-d]pyrimidin-4-yl)-1H-pyrazol-1-yl]propanenitrile; or a pharmaceutically acceptable salt thereof, for use in treating a hematological malignancy, characterized in that
i) a therapeutically effective amount of said compound or its pharmaceutically acceptable salt is administered to the patient on the basis of said patient having an increase in the level of expression of at least one or more biomarkers listed in Table 1 in a biological sample; or
ii) a therapeutically effective amount of another compound other than a STAT signaling inhibitor is administered to the patient on the basis of said patient having no increase in the level of expression of at least one or more biomarkers listed in Table 1 in a biological sample.

In any of the methods described herein the level of mRNA expression of any one, two, three, four, five, six, or seven biomarkers listed in Table 1 can be determined

### BRIEF DESCRIPTION OF THE DRAWINGS

FIG. 1 depicts a graph showing relationship between p-STAT5 status and 7-gene signature gene set activity scores across all haematopoietic cell lines.

FIG. 2A depicts a bar chart of pSTAT5 modulation by (R)-3-cyclopentyl-3-[4-(7H-pyrrolo[2,3-d]pyrimidin-4-yl)-1H-pyrazol-1-yl]propanenitrile and the effects on signature gene in RPMI 8226 (pSTAT5 negative cell line) and FIG. 2B depicts a bar chart of pSTAT5 modulation by (R)-3-cyclopentyl-3-[4-(7H-pyrrolo[2,3-d]pyrimidin-4-yl)-1H-pyrazol-1-yl]propanenitrileand and the effects on signature gene normalized expression in TF-1 (pSTAT5 positive cell line).

FIG. 3 depicts a bar chart showing pSTAT5 modulations in pSTAT5 positive cell lines by (R)-3-cyclopentyl-3-[4-(7H-pyrrolo[2,3-d]pyrimidin-4-yl)-1H-pyrazol-1-yl]propanenitrile at varying concentrations and the effects on signature genes in the cell line.

FIG. 4 depicts a bar chart showing modulations in pSTAT5 in pSTAT5 negative cell lines by (R)-3-cyclopentyl-3-[4-(7H-pyrrolo[2,3-d]pyrimidin-4-yl)-1H-pyrazol-1-yl]propanenitrile at varying concentrations and the effects on signature genes in the cell line.

FIG. 5 depicts a bar chart showing effects on signature genes in DMSO untreated pSTAT5 negative cell and positive cell lines at 4 hours.

FIG. 6 depicts a bar chart showing the 4 gene signature in UKE-1 tumor xenograft in vivo.

### DETAILED DESCRIPTION OF THE INVENTION

There is an increasing body of evidence that suggests a patient's genetic profile can be determinative to a patient's responsiveness to a therapeutic treatment. Given the numerous therapies available to treat cancer, a determination of the genetic factors that influence, for example, response to a particular drug, could be used to provide a patient with a personalized treatment regime. Such personalized treatment regimes offer the potential to maximize therapeutic benefit to the patient while minimizing related side effects that can be associated with alternative treatment regimes. Thus, there is a need to identify factors which can be used to predict whether a patient is likely to respond to a particular therapy.

To maximize the potential clinical benefit of a patient receiving a STAT signaling inhibitor it is important to be able to select those patients who have tumors that have an activated STAT signaling pathway. We have identified one or more biomarkers, the expression of which correlate significantly to the status of phosphorylation of STAT5. The present gene signature provides a reliable and easy-to-operate method to identify human cancers with activated STAT5 and identify cancers that would benefit from treatments targeting the STAT pathway such as the JAK/STAT pathway. If the subject has not been identified to have an activated STAT5, the subject should be administered with a non-JAK/STAT signaling molecule.

The methods described herein are based, in part, upon the identification of one or more of the biomarkers listed in Table 1, which can be used to determine if a patient would benefit from treatment with or administration of a therapeutically effective amount of a JAK/STAT inhibitor. The biomarkers of the invention were purposefully optimized for routine clinical testing.

The term “administering” in relation to a STAT signaling inhibitor, e.g., a JAK/STAT inhibitor, is used to refer to delivery of that compound to a patient by any route.

As used herein, a “therapeutically effective amount” refers to an amount of a STAT signaling inhibitor, e.g., a JAK/STAT inhibitor, that is effective, upon single or multiple dose administration to a patient (such as a human) for treating, preventing, preventing the onset of, curing, delaying, reducing the severity of, ameliorating at least one symptom of a disorder or recurring disorder, or prolonging the survival of the patient beyond that expected in the absence of such treatment. When applied to an individual active ingredient administered alone, the term refers to that ingredient alone. When applied to a combination, the term refers to combined amounts of the active ingredients that result in the therapeutic effect, whether administered in combination, serially or simultaneously.

The term “treatment” or “treat” refer to both prophylactic or preventative treatment (as the case may be) as well as curative or disease modifying treatment, including treatment of a patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment.

The phrase “respond to treatment” is used to mean that a patient, upon being delivered a particular treatment, e.g., a JAK/STAT inhibitor shows a clinically meaningful benefit from said treatment. The phrase “respond to treatment” is meant to be construed comparatively, rather than as an absolute response.

As used herein, “selecting” and “selected” in reference to a patient is used to mean that a particular patient is specifically chosen from a larger group of patients on the basis of (due to) the particular patient having a predetermined criteria, e.g., the patient has increased expression of at least one biomarker in Table 1. Similarly, “selectively treating” refers to providing treatment to a patient having a particular disease, where that patient is specifically chosen from a larger group of patients on the basis of the particular patient having a predetermined criteria, e.g., a haematological patient specifically chosen for treatment due to the patient having an increase in expression of a biomarker listed in Table 1. Similarly, “selectively administering” refers to administering a drug to a patient that is specifically chosen from a larger group of patients on the basis of (due to) the particular patient having a predetermined criteria, e.g., a patient having an increase in expression of a biomarker listed in Table 1. By selecting, selectively treating and selectively administering, it is meant that a patient is delivered a personalized therapy based on the patient's particular biology, rather than being delivered a standard treatment regimen based solely on the patient having a particular disease. Selecting, in reference to a method of treatment as used herein, does not refer to fortuitous treatment of a patient that has an increase in expression of a biomarker listed in Table 1, but rather refers to the deliberate choice to administer a JAK/STAT inhibitor to a patient based on the patient having patient having an increase in expression of a biomarker listed in Table 1. Thus, selective treatment differs from standard treatment, which delivers a particular drug to all patients, regardless of their biomarker expression status.

As used herein, “predicting” indicates that the methods described herein provide information to enable a health care provider to determine the likelihood that an individual having a haematological disease will respond to or will respond more favorably to treatment with a JAK/STAT inhibitor. It does not refer to the ability to predict response with 100% accuracy. Instead, the skilled artisan will understand that it refers to an increased probability.

As used herein, “likelihood” and “likely” is a measurement of how probable an event is to occur. It may be used interchangably with “probability”. Likelihood refers to a probability that is more than speculation, but less than certainty. Thus, an event is likely if a reasonable person using common sense, training or experience concludes that, given the circumstances, an event is probable. In some embodiments, once likelihood has been ascertained, the patient may be treated (or treatment continued, or treatment proceed with a dosage increase) with the JAK/STAT inhibitor or the patient may not be treated (or treatment discontinued, or treatment proceed with a lowered dose) with the JAK/STAT inhibitor.

The phrase “increased likelihood” refers to an increase in the probability that an event will occur. For example, some methods herein allow prediction of whether a patient will display an increased likelihood of responding to treatment with JAK/STAT inhibitor or an increased likelihood of responding better to treatment with JAK/STAT inhibitor based on an increased expression level of one or more biomarkers listed in Table 1 as compared to a patient which shows no increase in the expression level of one or more biomarkers listed in Table 1.

STAT Signaling Inhibitors

A STAT signaling inhibitor used in the present invention can include any molecule that directly or indirectly inhibits the STAT signaling pathway resulting in a decrease in phosphorylation of one or more STAT proteins. Such inhibitors can include JAK inhibitors (otherwise referred to herein as JAK/STAT inhibitors), ALK inhibitors (otherwise referred to herein as ALK/STAT inhibitors), EGFR inhibitors (otherwise referred to herein as EGFR/STAT inhibitors), or a SRK inhibitor (otherwise referred to herein as SRK/STAT inhibitors).

A JAK/STAT inhibitor is any compound that selectively inhibits the activity of any JAK molecule such as JAK 1, 2, 3, and 4 or any STAT molecule such as STAT 3 and STATS. In one example, the JAK/STAT inhibitor is a JAK2 inhibitor. JAK2 inhibitors are known in the art, and include for example small molecule compounds, small peptides, antibodies, antisense oligonucleotides, siRNAs, and the like. In some embodiments, the JAK2 inhibitor can be INCB018424, XL019, TG101348, or TG101209.

In one embodiment, the JAK2 inhibitor is a compound of Formula I:

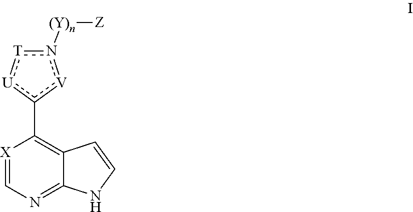

or a pharmaceutically acceptable salt thereof, wherein:

T, U, and V are independently selected from O, S, N, CR⁵, and NR⁶;

wherein the 5-membered ring formed by carbon atom, nitrogen atom, U, T, and V is aromatic; X is N or CR⁴;

n is 0; or

n is 1 and Y is C₁₋₈ alkylene, C₂₋₈ alkenylene, (CR¹¹R¹²)ₚC(O)(CR¹¹R¹²)_{q}, (CR₁₁R¹²)ₚC(O)NR^{c}(CR¹¹R¹²)_{q}, (CR¹¹R¹²)ₚC(O)O(CR¹¹R¹²)_{q}, or (CR¹¹R¹²)ₚOC(O)(CR¹¹R¹²)_{q},

wherein said C₁₋₈ alkylene or C₂₋₈ alkenylene, is optionally substituted with 1, 2, or 3 halo, OH, CN, amino, C₁₋₄ alkylamino, or C₂₋₈ dialkylamino;

Z is aryl, cycloalkyl, heteroaryl, or heterocycloalkyl, each optionally substituted with 1, 2, 3, 4, 5, or 6 independently substituents selected from halo, C₁₋₄ alkyl, C₂₋₄ alkenyl, C₂₋₄ alkynyl, C₁₋₄ haloalkyl, C₁₋₄ hydroxyalkyl, C₁₋₄ cyanoalkyl, Cy¹, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, S(O)R^{b}, S(O)NR^{c}R^{d}, S(O)₂R^{b}, NR^{c}S(O)₂R^{b}, and S(O)₂NR^{c}R^{d};

Cy¹ is independently selected from aryl, heteroaryl, cycloalkyl, and heterocycloalkyl, each optionally substituted by 1, 2, 3, 4 or 5 substituents independently selected from halo, C₁₋₄ alkyl, C₂₋₄ alkenyl, C₂₋₄ alkynyl, C₁₋₄ haloalkyl, CN, NO₂, OR^{a″}, SR^{a″}, C(O)R^{b″}, C(O)NR^{c″}R^{d″}, C(O)OR^{a″}, OC(O)R^{b″}, OC(O)NR^{c″}R^{d″}, NR^{c″}R^{d″}, NR^{c″}C(O)R^{b″}, NR^{c″}C(O)OR^{a″}, S(O)R^{b″}, S(O)NR^{c″}R^{d″}, S(O)₂R^{b″}, and S(O)₂NR^{c″}R^{d″};

R⁴ is H;

R⁵ is H, halo, C₁₋₄ alkyl, C₂₋₄ alkenyl, C₂₋₄ alkynyl, C₁₋₄ haloalkyl, CN, NO₂, OR⁷, SR⁷, C(O)R⁸, C(O)NR⁹R¹⁰, C(O)OR⁷, OC(O)R⁸, OC(O)NR⁹R¹⁰, NR⁹R¹⁰, NR⁹C(O)R⁸, NR⁹C(O)OR⁷, S(O)R⁸, S(O)NR⁹R¹⁰, S(O)₂R⁸, NR⁹S(O)₂R⁸, or S(O)₂NR⁹R¹⁰;

R⁶ is H, C₁₋₄ alkyl, C₂₋₄ alkenyl, C₂₋₄ alkynyl, C₁₋₄ haloalkyl, OR⁷, C(O)R⁸, C(O)NR⁹R¹⁰, C(O)OR⁷, S(O)R⁸, S(O)NR⁹R¹⁰, S(O)₂R⁸, or S(O)₂NR⁹R¹⁰;
R⁷ is H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl;
R⁸ is H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl;
R⁹ and R¹⁰ are independently selected from H, C₁₋₁₀ alkyl, C₁₋₆ haloalkyl, C₂₋₆ alkenyl, C₂₋₆ alkynyl, C₁₋₆ alkylcarbonyl, arylcarbonyl, C₁₋₆ alkylsulfonyl, arylsulfonyl, aryl, heteroaryl, cycloalkyl, heterocycloalkyl, arylalkyl, heteroarylalkyl, cycloalkylalkyl and heterocycloalkylalkyl;
or R⁹ and R¹⁰ together with the N atom to which they are attached form a 4-, 5-, 6- or 7-membered heterocycloalkyl group;
R¹¹ and R¹² are independently selected from H, halo, OH, CN, C₁₋₄ alkyl, C₁₋₄ haloalkyl, C₂₋₄ alkenyl, C₂₋₄ alkynyl, C₁₋₄ hydroxyalkyl, C₁₋₄ cyanoalkyl, aryl, heteroaryl, cycloalkyl, and heterocycloalkyl;
R^{a} and R^{a″} are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl, wherein said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with 1, 2, or 3 substituents independently selected from OH, CN, amino, halo, C₁₋₆ alkyl, C₁₋₆ haloalkyl, aryl, arylalkyl, heteroaryl, heteroarylalkyl, cycloalkyl and heterocycloalkyl;
R^{b} and R^{b″} are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl, wherein said C₁₋₆ alkyl, C₁₋₆ haloalkyl, C₂₋₆ alkenyl, C₂₋₆ alkynyl, aryl, cyclo-alkyl, heteroaryl, heterocycloalkyl, arylalkyl, heteroarylalkyl, cycloalkylalkyl or heterocycloalkylalkyl is optionally substituted with 1, 2, or 3 substituents independently selected from OH, CN, amino, halo, C₁₋₆ alkyl, C₁₋₆ haloalkyl, C₁₋₆ haloalkyl, aryl, arylalkyl, heteroaryl, heteroarylalkyl, cycloalkyl and heterocycloalkyl;
R^{c} and R^{d} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein said C₁₋₁₀ alkyl, C₁₋₆ haloalkyl, C₂₋₆ alkenyl, C₂₋₆ alkynyl, aryl, hetero-aryl, cycloalkyl, heterocycloalkyl, arylalkyl, heteroarylalkyl, cycloalkylalkyl or heterocycloalkylalkyl is optionally substituted with 1, 2, or 3 substituents independently selected from OH, CN, amino, halo, C₁₋₆ alkyl, C₁₋₆haloalkyl, C₁₋₆haloalkyl, aryl, arylalkyl, heteroaryl, heteroarylalkyl, cycloalkyl or heterocycloalkyl;
or R^{c} and R^{d} together with the N atom to which they are attached form a 4-, 5-, 6- or 7-membered heterocycloalkyl group optionally substituted with 1, 2, or 3 substituents independently selected from OH, CN, amino, halo, C₁₋₆ alkyl, C₁₋₆ haloalkyl, C₁₋₆ haloalkyl, aryl, arylalkyl, heteroaryl, heteroarylalkyl, cycloalkyl and heterocycloalkyl;
R^{c″} and R^{d} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein said C₁₋₁₀ alkyl, C₁₋₆ haloalkyl, C₂₋₆ alkenyl, C₂₋₆ alkynyl, aryl, hetero-aryl, cycloalkyl, heterocycloalkyl, arylalkyl, heteroarylalkyl, cycloalkylalkyl or heterocycloalkylalkyl is optionally substituted with 1, 2, or 3 substituents independently selected from OH, CN, amino, halo, C₁₋₆ alkyl, C₁₋₆ haloalkyl, C₁₋₆ haloalkyl, aryl, arylalkyl, heteroaryl, heteroarylalkyl, cycloalkyl and heterocycloalkyl;
or R^{c″} and R^{d″}together with the N atom to which they are attached form a 4-, 5-, 6- or 7-membered heterocycloalkyl group optionally substituted with 1, 2, or 3 substituents independently selected from OH, CN, amino, halo, C₁₋₆ alkyl, C₁₋₆ haloalkyl, C₁₋₆ haloalkyl, aryl, arylalkyl, heteroaryl, heteroarylalkyl, cycloalkyl and heterocycloalkyl;

p is 0, 1, 2, 3, 4, 5, or 6; and

q is 0, 1, 2, 3, 4, 5 or 6.

In a particular embodiment, the JAK2 inhibitor is 3-cyclopentyl-3-[4-(7H-pyrrolo[2,3-d]pyrimidin-4-yl)-1H-pyrazol-1-yl]propanenitrile; or a pharmaceutically acceptable salt thereof. In another embodiment, the compound is (R)-3-cyclopentyl-3-[4-(7H-pyrrolo[2,3-d]pyrimidin-4-yl)-1H-pyrazol-1-yl]propanenitrile; or a pharmaceutically acceptable salt thereof.

Biomarker

The biomarker(s) of the invention includes one or more genes, such as any 1, 2, 3, 4, 5, 6 or 7 genes listed in Table 1. By analyzing the mRNA expression level of one or more biomarkers identified in Table 1 it is possible to select individuals having cancers in which the JAK/STAT pathway is activated and who thus are likely to respond to treatment with an inhibitor of the JAK/STAT signaling pathway, e.g., a JAK2 inhibitor.

TABLE 1

| | Accession # Uni |
|---|---|
| Gene Name | Gene ID |
| Pim-1 oncogene (PIM 1) | 5292 |
| Cytokine inducible SH2-containing | 1154 |
| protein (CISH) | |
| Suppressor of cytokine signaling | 8835 |
| 2 (SOCS2) | |
| Inhibitor of DNA binding 1, dominant | 3397 |
| negative helix-loop-helix protein (ID1) | |
| Lipocalin 2 (LCN2) | 3934 |
| Erythropoietin receptor (EPOR) | 2057 |
| Early growth response 1 (EGR1) | 1958 |

In addition, the level of expression of a house keeping gene or a normalization gene contained within the sample can be determined for RT-PCR. In one example, the house keeping gene to be used in the present invention can be glucuronidase, beta (GUSB; UGID:170831; UniGeneHs.255230) and/or TATA-binding protein (TBP; Accession Uni Gene ID UGID:2059883; UniGene Hs.590872).

Preparation of Samples

Any appropriate test sample of cells taken from an individual having a proliferative disease can be used. Generally, the test sample of cells or tissue sample will be obtained from the subject with cancer by biopsy or surgical resection. A sample of cells, tissue, or fluid may be removed by needle aspiration biopsy. For this, a fine needle attached to a syringe is inserted through the skin and into the tissue of interest. The needle is typically guided to the region of interest using ultrasound or computed tomography (CT) imaging. Once the needle is inserted into the tissue, a vacuum is created with the syringe such that cells or fluid may be sucked through the needle and collected in the syringe. A sample of cells or tissue may also be removed by incisional or core biopsy. For this, a cone, a cylinder, or a tiny bit of tissue is removed from the region of interest. CT imaging, ultrasound, or an endoscope is generally used to guide this type of biopsy. More particularly, the entire cancerous lesion may be removed by excisional biopsy or surgical resection. In the present invention, the test sample is typically a sample of cells removed as part of surgical resection.

The test sample of, for example tissue, may also be stored in, e.g., RNAlater (Ambion; Austin Tex.) or flash frozen and stored at −80° C. for later use. The biopsied tissue sample may also be fixed with a fixative, such as formaldehyde, paraformaldehyde, or acetic acid/ethanol. The fixed tissue sample may be embedded in wax (paraffin) or a plastic resin. The embedded tissue sample (or frozen tissue sample) may be cut into thin sections. RNA or protein may also be extracted from a fixed or wax-embedded tissue sample or a frozen tissue sample. Once a sample of cells or sample of tissue is removed from the subject with cancer, it may be processed for the isolation of RNA or protein using techniques well known in the art and as described below.

An example of extraction of RNA from a biopsy taken from a patient with cancers can include, for example, guanidium thiocyanate lysis followed by CsCl centrifugation (Chirgwin, et al., Biochemistry 18:5294-5299, 1979). RNA from single cells may be obtained as described in methods for preparing cDNA libraries from single cells (see, e.g., Dulac, Curr. Top. Dev. Biol. 36:245, 1998; Jena, et al., J. Immunol. Methods 190:199, 1996). In one embodiment, the RNA population may be enriched for sequences of interest, as detailed in Table 1. Enrichment may be accomplished, for example, by random hexamers and primer-specific cDNA synthesis, or multiple rounds of linear amplification based on cDNA synthesis and template-directed in vitro transcription (see, e.g., Wang, et al., Proc. Natl. Acad. Sci. USA 86:9717, 1989; Dulac, et al., supra; Jena, et al., supra).

The JAK/STAT expression profile can be performed on a biopsy taken from a subject such as fresh tissue, frozen tissue, tissue processed in formalin (FFPE) or other fixatives.

The subject with a tumor or cancer will generally be a mammalian subject such as a primate. In an exemplary embodiment, the subject is a human.

Any cancer or tumor can be screened according to the methods of the invention and include, but are not limited to, hematological malignancies, ovarian colon cancer, lung cancer, pancreatic cancer, gastric cancer, prostate cancer, and hepatocellular carcinoma, basal cell carcinoma, breast cancer, bone sarcoma, soft tissue sarcoma, medulloblastoma, rhabdomyosaracoma, neuroblastoma, pancreatic cancer, meningioma, glioblastoma, astrocytoma, melanoma, stomach cancer, esophageal cancer, biliary tract cancer, small cell lung cancer, non-small cell lung cancer, glial cell cancer, multiple myeloma, colon cancer, neuroectodermal tumor, neuroendocrine tumor, mastocytoma and Gorlin syndrome.

In particular the invention can be used to treat patients who have hematological malignancies such as leukemia, lymphomas and myelomas. In one example, the leukemia is Acute lymphoblastic leukemia (ALL), Acute myelogenous leukemia (AML), Chronic lymphocytic leukemia (CLL), Chronic myelogenous leukemia (CML), Chronic myelogenous leukemia (CML), or Acute monocytic leukemia (AMOL). In another embodiment of the invention, the hematological malignancy is polycythemia vera (PV), essential thrombocythemia (ET), myeloid metaplasia with myelofibrosis (MMM), chronic myelomonocytic leukemia (CMML), hypereosinophilic syndrome (HES), or systemic mast cell disease (SMCD). In another example, the lymphoma is Hodgkin's lymphomas or non-Hodgkin's lymphoma.

Detection of Expression of the Biomarker

In one example, the method includes determining expression of one or more of the genes of Table 1. The gene sequences of interest can be detected using agents that can be used to specifically detect the gene, for example, RNA transcribed from the gene.

Analysis of the sequence of mRNA transcribed from a given biomarker can be performed using any known method in the art including, but not limited, to Northern blot analysis, nuclease protection assays (NPA), in situ hybridization, reverse transcription-polymerase chain reaction (RT-PCR), RT-PCR ELISA, TaqMan-based quantitative RT-PCR (probe-based quantitative RT-PCR) and SYBR green-based quantitative RT-PCR. In one example, detection of mRNA levels involves contacting the isolated mRNA with an oligonucleotide that can hybridize to mRNA. The nucleic acid probe can typically be, for example, a full-length cDNA, or a portion thereof, such as an oligonucleotide of at least 7, 15, 30, 50, or 100 nucleotides in length and sufficient to specifically hybridize under stringent conditions to the mRNA of interest, e.g., mRNA of one or more of the genes listed in Table 1. In one format, the RNA is immobilized on a solid surface and contacted with a probe, for example by running the isolated RNA on an agarose gel and transferring the mRNA from the gel to a membrane, such as nitrocellulose. Amplification primers are defined as being a pair of nucleic acid molecules that can anneal to 5′ or 3′ regions of a biomarker gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers. PCR products can be detected by any suitable method including, but not limited to, gel electrophoresis and staining with a DNA-specific stain or hybridization to a labeled probe.

The level of expression of a biomarker may be determined by measuring RNA (or reverse transcribed cDNA) levels using various techniques, e.g., a PCR-based assay, reverse-transcriptase PCR (RT-PCR) assay, Northern blot, etc. Quantitative RT-PCR with standardized mixtures of competitive templates can also be utilized.

In one embodiment, the method includes: providing a nucleic acid probe comprising a nucleotide sequence, for example, at least 7, 10, 15, 20, 25, 30 or 40 nucleotides, and up to all or nearly all of the coding sequence which is complementary to a portion of the coding sequence of a nucleic acid sequence of any one or more of the genes of Table 1; obtaining a tissue sample from a mammal having a cancerous cell; contacting the nucleic acid probe under stringent conditions with RNA obtained from a biopsy taken from a patient with cancer (e.g., in a Northern blot, in situ hybridization assay, PCR etc); and determining the amount of hybridization of the probe with RNA. Nucleic acids may be labeled during or after enrichment and/or amplification of RNAs.

The biomarkers of Table 1 are intended to also include naturally occurring sequences including allelic variants and other family members. The biomarkers of the invention also include sequences that are complementary to those listed sequences resulting from the degeneracy of the code and also sequences that are sufficiently homologous and sequences which hybridize under stringent conditions to the genes of the invention.

By “sufficiently homologous” it is meant a amino acid or nucleotide sequence of a biomarker which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains or motifs and/or a common functional activity. For example, amino acid or nucleotide sequences which share common structural domains have at least about 50 percent homology, at least about 60 percent homology, at least about 70 percent, at least about 80 percent, and at least about 90-95 percent homology across the amino acid sequences of the domains are defined herein as sufficiently homologous. Furthermore, amino acid or nucleotide sequences at least about 50 percent homology, at least about 60-70 percent homology, at least about 70-80 percent, at least about 80-90 percent, and at least about 90-95 percent and share a common functional activity are defined herein as sufficiently homologous.

The comparison of sequences and determination of percent homology between two sequences can be accomplished using a mathematical algorithim. A preferred, non-limiting example of a mathematical algorithim utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to TRL nucleic acid molecules of the invention. BLAST protein searches can be performed with the XBLAST program, score=50, wordlength=3 to obtain amino acid sequences homologous to the protein sequences encoded by the genes listed in Table 1. To obtain gapped alignments for comparison purposes, Gapped BLAST can be utilized as described in Altschul et al., (1997) Nucleic Acids Research 25(17):3389-3402. When utilizing BLAST and Gapped BLAST programs, the default parameters of the respective programs (e.g., XBLAST and NBLAST) can be used. See http://www.ncbi.nlm.nih.gov. Another preferred, non-limiting example of a mathematical algorithim utilized for the comparison of sequences is the ALIGN algorithm of Myers and Miller, CABIOS (1989). When utilizing the ALIGN program for comparing amino acid sequences, a PAM1 20 weight residue table, a gap length penalty of 12, and a gap penalty of 4 can be used.

The term “probe” refers to any composition of matter that is useful for specifically detecting another substance. In preferred embodiments, the probe specifically hybridizes to a nucleic acid sequence (preferably genomic DNA) or specifically binds to a polypeptide sequence of an allele of interest. The phrase “specifically hybridizes” is used to refer to hybrization under stringent hybridization conditions. Stringent conditions are known to those skilled in the art and can be found in Current Protocols in Molecular Biology, John Wiley & Sons, N.Y. (1989), 6.3.1-6.3.6. Aqueous and nonaqueous methods are described in that reference and either can be used. One example of stringent hybridization conditions is hybridization in 6× sodium chloride/sodium citrate (SSC) at about 45° C., followed by at least one wash in 0.2×SSC, 0.1% SDS at 50° C. A second example of stringent hybridization conditions is hybridization in 6×SSC at about 45° C., followed by at least one wash in 0.2×SSC, 0.1% SDS at 55° C. Another example of stringent hybridization conditions is hybridization in 6×SSC at about 45° C., followed by at least one wash in 0.2×SSC, 0.1% SDS at 60° C. A further example of stringent hybridization conditions is hybridization in 6×SSC at about 45° C., followed by at least one wash in 0.2×SSC, 0.1% SDS at 65° C. High stringent conditions include hybridization in 0.5 M sodium phosphate, 7% SDS at 65° C., followed by at least one wash at 0.2×SSC, 1% SDS at 65° C.

An “oliogonucelotide” refers to a short sequence of nucleotides, e.g., 2-100 bases.

The present invention includes measuring the expression of one or more genes PIM1, CISH SOCS2, ID1, LCN2, EPOR and EGR1 in a tumor biopsy taken from a subject suffering from cancer, e.g., haematological disorder, due to JAK/STAT pathway activation. The expression levels can be analyzed and used to generate a score which can be used to differentiate those patients having a tumor exhibiting JAK/STAT pathway activation versus those who do not.

In one embodiment, the method of the invention includes measuring the expression of any one of PIM1, CISH SOCS2, ID1, LCN2, EPOR and EGR1 listed in Table 1. In another embodiment, the method of the invention includes measuring at least one e.g., at least two, at least three, at least four, at least five, at least six, or at least seven from Table 1.

In one example, the level of expression of one gene, e.g., PIM-1, from Table 1 is measured. In another example, the level of expression of two genes, e.g., PIM1 and CISH, from Table 1 is measured. In yet another example, the level of expression of three genes PIM1, CISH and SOCS2 from Table 1 is measured. In yet another example, the level of expression of four genes PIM1, CISH SOCS2, and ID 1 from Table 1 is measured. In yet another example, the level of expression of five genes PIM1, CISH SOCS2, ID1, and LCN2 from Table 1 is measured. In yet another example, the level of expression of six genes PIM1, CISH SOCS2, ID1, LCN2 and EPOR. In yet another example, the level of expression of seven genes PIM1, CISH SOCS2, ID1, LCN2, EPOR and EGR1.

The biomarkers of the invention also include any combination of genes identified in Table 1 whose level of expression or gene product serves as a predictive marker or biomarker.

In the method of the invention the level of expression of one or more genes as described above is measured and analyzed and used to generate a score which can be used to select those subjects having a tumor due to JAK/STAT pathway activation as described below. The expression threshold can be used to select for those individuals who have will respond to a JAK/STAT inhibitor.

It is necessary to normalize differences in the amount of RNA assayed and variability in the quality of the RNA used. Therefore, the assay typically measures and incorporates the expression of certain normalizing genes.

In the methods of the invention, the expression of each biomarker is measured and typically will be converted into an expression value after normalization by the expression level of a control gene. These expression values then will be used to generate a score which is then compared against a cut-off to select which subjects have a JAK/STAT-activated tumor and therefore are likely to benefit from treatment with a JAK/STAT inhibitor.

The biomarkers of the invention can be measured using any method known in the art such as reverse Transcriptase PCR (RT-PCR). The method includes isolating mRNA using any technique known in the art, e.g., by using a purification kit, buffer set and protease from commercial manufacturers, such as Qiagen. The reverse transcription step is typically primed using specific primers, random hexamers, or oligo-dT primers, depending on the circumstances and the goal of expression profiling and the cDNA derived can then be used as a template in the subsequent PCR reaction. TaqMan(R) RT-PCR can then be performed using, e.g., commercially available equipment.

A more recent variation of the RT-PCR technique is the real time quantitative PCR, which measures PCR product accumulation through a dual-labeled fluorigenic probe (e.g., using TaqMan(R) probe). Real time PCR is compatible both with quantitative competitive PCR, where internal competitor for each target sequence is used for normalization, and with quantitative comparative PCR using a normalization gene contained within the sample, or a housekeeping gene for RT-PCR. For further details see, e.g. Held et al, Genome Research 6:986-994 (1996).

In another example, microarrays are used which include one or more probes corresponding to one or more of genes of Table 1. The method described above results in the production of hybridization patterns of labeled target nucleic acids on the array surface. The resultant hybridization patterns of labeled nucleic acids may be visualized or detected in a variety of ways, with the particular manner of detection selected based on the particular label of the target nucleic acid. Representative detection means include scintillation counting, autoradiography, fluorescence measurement, calorimetric measurement, light emission measurement, light scattering, and the like.

In another example, a TaqMan® Low Density Array (TLDA) card can be used which can include one or more probes corresponding to one or more of genes of Table 1. This method uses a microfluidic card that performs simultaneous real time PCR reactions.

In one example, the method of detection utilizes an array scanner that is commercially available (Affymetrix, Santa Clara, Calif.), for example, the 417 Arrayer, the 418 Array Scanner, or the Agilent GeneArray Scanner. This scanner is controlled from a system computer with an interface and easy-to-use software tools. The output may be directly imported into or directly read by a variety of software applications. Scanning devices are described in, for example, U.S. Pat. Nos. 5,143,854 and 5,424,186.

In yet another example, mRNA levels can be analyzed using expression analysis of high-throughput mRNA sequencing (RNA-seq). Examples of useful platforms that can be used to study mRNA expression levels include Illumina sequencing (formerly Solexa sequencing) platform.

As used herein, the control for comparison can be determined by one skilled in the art. In one aspect, the control is determined by choosing a value that serves as a cut-off value. For example, the value can be a value that differentiates between e.g., those test samples that have JAK/STAT activation (phosphorylated STAT5+) from those that do not show JAK/STAT activation (no phosphorylation of STAT5). In another example, the gene expression profile of a biomarker of the invention is compared to a control (presence of expression of the biomarker in a sample taken from a healthy person or a tumor that is JAK/STAT-activated).

Data Analysis

To facilitate the sample analysis operation, the data obtained by the reader from the device may be analyzed using a digital computer. Typically, the computer will be appropriately programmed for receipt and storage of the data from the device, as well as for analysis and reporting of the data gathered, for example, subtraction of the background, verifying that controls have performed properly, normalizing the signals, interpreting fluorescence data to determine the amount of hybridized target, normalization of background, and the like.

In one example, once the level of expression of one or more markers in Table 1 is determined, physicians or genetic counselors or patients or other researchers may be informed of the result. Specifically the result can be cast in a transmittable form of information that can be communicated or transmitted to other researchers or physicians or genetic counselors or patients. Such a form can vary and can be tangible or intangible. The result in the individual tested can be embodied in descriptive statements, diagrams, photographs, charts, images or any other visual forms. For example, images of gel electrophoresis of PCR products can be used in explaining the results. Diagrams showing levels of biomarker expression are also useful in indicating the testing results. These statements and visual forms can be recorded on a tangible media such as papers, computer readable media such as floppy disks, compact disks, etc., or on an intangible media, e.g., an electronic media in the form of email or website on internet or intranet. In addition, the result can also be recorded in a sound form and transmitted through any suitable media, e.g., analog or digital cable lines, fiber optic cables, etc., via telephone, facsimile, wireless mobile phone, internet phone and the like. All such forms (tangible and intangible) would constitute a “transmittable form of information”. Thus, the information and data on a test result can be produced anywhere in the world and transmitted to a different location. For example, when the assay is conducted offshore, the information and data on a test result may be generated and cast in a transmittable form as described above. The test result in a transmittable form thus can be imported into the U.S. Accordingly, the present disclosure also encompasses a method for producing a transmittable form of information containing levels of expression of biomarkers listed in Table 1. This form of information is useful for predicting the responsiveness of a patient to treatment with a JAK/STAT inhibitor, for selecting a course of treatment based upon that information, and for selectively treating a patient based upon that information.

Kits

The invention further provides kits for determining the expression level of the biomarkers described herein. The kits may be useful for determining who will benefit from treatment with a JAK/STAT inhibitor. A kit can comprise probes/oligonucleotides/primers of genes identified in Table 1 can be used to measure gene expression of a test sample. In one embodiment, the kit comprises a computer readable medium which includes expression profile analysis software capable of being loaded into the memory of a computer system and which can convert the measured expression values into a risk score. A kit may further comprise nucleic acid controls, buffers, and instructions for use.

Administration

The STAT signaling inhibitors described herein can be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

### EXAMPLES

### Examples

### Example 1

### Generation of Gene Signature

In order to identify a mRNA expression based signature to discriminate p-STAT5 positive and p-STAT5 negative samples we used two sets of haematopoietic cell lines with p-STAT5 western blot data. Each independent set has mRNA expression profile data from the Affymetrix U133Plus2 arrays. All expression values are MAS5 normalized, with a 2% trimmed mean of 150.

The first set has data for 28 cell lines with 8 p-STAT5 positive and 20 p-STAT5 negative (by western). This was used as the signature-enrichment set. The second set has data for 12 unique cell lines, with 6 p-STAT5 positive and 6 p-STAT5 negative (by western). The samples unique in set 2 were used as the signature validation set.

The pSTAT5 status from sets 1 and 2 are summarized in Table 2.

TABLE 2

| Cell Line Name | pSTAT5 set 1 | pSTAT5 set 2 |
|---|---|---|
| THP-1 | N | |
| MM1-S | N | |
| ST486 | N | |
| NCI-H929 | N | |
| JM1 | N | |
| Loucy | N | |
| RPMI 8226 | N | |
| Toledo | N | |
| MC116 | N | |
| Reh | N | |
| KMS-12-BM | N | |
| RS4; 11 | N | |
| BDCM | N | |
| U-937 | N | |
| HD-MY-Z | N | |
| HuNS1 | N | |
| SUP-T1 | N | |
| CA46 | N | |
| RL | N | |
| HH | N | |
| MOLM-13 | Y | |
| AML-193 | Y | |
| Set-2 | Y | |
| TF-1 | Y | |
| HEL 92.1.7 | Y | |
| K-562 | Y | |
| SUP-B15 | Y | |
| MEG-01 | Y | |
| PL-21 | | N |
| OCI-AML2 | | N |
| NOMO-1 | | N |
| HL-60 | | N |
| KASUMI-1 | | N |
| SKM-1 | | N |
| EOL-1 | | Y |
| F-36P | | Y |
| Kasumi-6 | | Y |
| MV-4-11 | | Y |
| M-07e | | Y |
| OCI-AML5 | | Y |

We selected 47 genes which are considered to be transcriptional targets of STAT5 and have probe sets on the U133Plus2 array (MetaCore from GeneGo Inc.). For each of the 47 genes, the best probe set was chosen based on combination of manual review and computational approach. The approach for selecting the best probe set per gene is regularly used for analysis of Affymetrix gene expression data, and the list of best probe sets was determined independently of this project.

For each of 47 genes, the fold change and probability associated between p-STAT5 positive and p-STAT5 negative cell lines was calculated with the Student's t-Test using data from the enrichment cell line set. For fold change calculations, a value of 50 was added to the expression averages for p-STAT5 positive and p-STAT5 negative cell lines in order to decrease noise from low expressing genes. Positive values indicate higher expression in p-STAT5 positive lines, while negative values indicate higher expression in p-STAT5 negative lines. Student's t-Test was run using two-tailed distribution and homoscedastic settings. Table 2 provides the results for all 47 genes.

We used data from Table 3 to create 3 gene sets (Table 4). The first one included 4 genes (PIM1, CISH, SOCS2, ID1) with lowest p-values and fold changes above 4. The second gene set contains the aforementioned 4 genes and LCN2 and EPOR, both of which have fold changes around 2 and p-values below 0.01. The third gene set carries the additional gene, EGR1, which has fold change around 2.5, but p-value ˜0.06. Also included in the analysis is the 47-gene set.

TABLE 3

| | Entrez | | p-STAT5+ | p-STAT5− | | t-test p- |
|---|---|---|---|---|---|---|
| Gene Name | GeneID | probe set | mean | mean | fold | value |
| PIM1 | 5292 | 209193_at | 875 | 134 | 5.04 | 6.82E−07 |
| CISH | 1154 | 223961_s_at | 245 | 21 | 4.15 | 5.86E−06 |
| SOCS2 | 8835 | 203373_at | 2441 | 326 | 6.63 | 1.64E−05 |
| ID1 | 3397 | 208937_s_at | 1548 | 332 | 4.19 | 0.00331972 |
| LCN2 | 3934 | 212531_at | 80 | 8 | 2.24 | 0.00453474 |
| EPOR | 2057 | 209962_at | 118 | 38 | 1.91 | 0.00836353 |
| KIR3DL1 | 3811 | 211687_x_at | 24 | 14 | 1.15 | 0.02315812 |
| C3AR1 | 719 | 209906_at | 91 | 35 | 1.66 | 0.02897651 |
| BCL2L1 | 598 | 212312_at | 270 | 167 | 1.47 | 0.03413896 |
| IGJ | 3512 | 212592_at | 106 | 3746 | −24.29 | 0.04997906 |
| EGR1 | 1958 | 227404_s_at | 1035 | 351 | 2.71 | 0.0638939 |
| OSM | 5008 | 230170_at | 53 | 17 | 1.55 | 0.10218279 |
| TBX21 | 30009 | 220684_at | 40 | 12 | 1.46 | 0.14215803 |
| TNFRSF13B | 23495 | 207641_at | 27 | 71 | −1.57 | 0.15316237 |
| ESR1 | 2099 | 205225_at | 10 | 18 | −1.15 | 0.15905403 |
| XIAP | 331 | 228363_at | 711 | 1041 | −1.43 | 0.20670021 |
| ABCB1 | 5243 | 243951_at | 34 | 19 | 1.21 | 0.21057215 |
| IL18 | 3606 | 206295_at | 91 | 50 | 1.41 | 0.26985569 |
| SKP2 | 6502 | 210567_s_at | 256 | 345 | −1.29 | 0.27693167 |
| MYC | 4609 | 202431_s_at | 5556 | 4662 | 1.19 | 0.30379619 |
| SRP9 | 6726 | 201273_s_at | 5997 | 6579 | −1.1 | 0.36038668 |
| FOS | 2353 | 209189_at | 98 | 55 | 1.41 | 0.42764108 |
| IL10 | 3586 | 207433_at | 7 | 23 | −1.29 | 0.45530643 |
| EBF1 | 1879 | 227646_at | 565 | 1033 | −1.76 | 0.46111412 |
| CSN1S1 | 1446 | 208350_at | 4 | 3 | 1.02 | 0.50498373 |
| ONECUT1 | 3175 | 210745_at | 8 | 10 | −1.03 | 0.54105495 |
| HSD3B2 | 3284 | 206294_at | 4 | 5 | −1.02 | 0.54197609 |
| SLC30A2 | 7780 | 230084_at | 16 | 15 | 1.02 | 0.54712739 |
| SP1 | 6667 | 224760_at | 367 | 311 | 1.15 | 0.55826135 |
| PRF1 | 5551 | 214617_at | 76 | 73 | 1.02 | 0.56205153 |
| IFNG | 3458 | 210354_at | 8 | 9 | −1.03 | 0.56455525 |
| IL22 | 50616 | 222974_at | 6 | 4 | 1.02 | 0.56529166 |
| CITED4 | 163732 | 228625_at | 38 | 94 | −1.64 | 0.58702634 |
| CCND1 | 595 | 208712_at | 40 | 107 | −1.75 | 0.60420565 |
| RAD51 | 5888 | 205024_s_at | 576 | 626 | −1.08 | 0.66382789 |
| PAX5 | 5079 | 206802_at | 9 | 11 | −1.03 | 0.68588032 |
| CSN2 | 1447 | 207951_at | 10 | 11 | −1.02 | 0.69349354 |
| SOCS1 | 8651 | 210001_s_at | 142 | 102 | 1.26 | 0.72728647 |
| RBMS1 | 5937 | 225265_at | 310 | 296 | 1.04 | 0.7600465 |
| PTGS2 | 5743 | 204748_at | 28 | 49 | −1.27 | 0.78891958 |
| SOCS3 | 9021 | 227697_at | 118 | 26 | 2.21 | 0.81490784 |
| EPAS1 | 2034 | 200878_at | 429 | 157 | 2.31 | 0.8417473 |
| TRGC2 | 6967 | 216920_s_at | 466 | 410 | 1.12 | 0.8773761 |
| FOXP3 | 50943 | 221333_at | 3 | 3 | −1 | 0.93339042 |
| CDKN1A | 1026 | 202284_s_at | 173 | 182 | −1.04 | 0.94107376 |
| TLR2 | 7097 | 204924_at | 64 | 72 | −1.07 | 0.96066244 |
| GADD45G | 10912 | 204121_at | 11 | 11 | 1 | 0.98495784 |

TABLE 4

| 4-gene signature | 6-gene signature | 7-gene signature |
|---|---|---|
| PIM1 | PIM1 | PIM1 |
| CISH | CISH | CISH |
| SOCS2 | SOCS2 | SOCS2 |
| ID1 | ID1 | ID1 |
| | LCN2 | LCN2 |
| | EPOR | EPOR |
| | | EGR1 |

We used the validation set of cell lines to independently validate these gene sets. In order to do so we calculated gene set activity scores for each gene set. The approach for calculating gene set activity scores is regularly used for analysis of gene expression data, and was created independently of this project (Breslin T et al., 2005 BMC Bioinformatics. 6:163; Lee E et al., PLoS Comput. Biol. 2008; 4:e1000217; Guo Z et al., et al. 2005 BMC Bioinformatics. 2005; 6 :58.). Gene set activity score calculation is done in a 2 step process.

First step is to perform z-score transformation for each probe expression values across set of samples.

Zi,j=(Xi,j−μ)/(δ+ε)

Xi,j is MASS expression value for probe i in sample j

ε is standard Deviation Constant, 10 is used for MAS5 expression values. Second step is to calculate gene set activity scores by adding up Zi,j score from genes in particular gene set and normalizing by square root of number genes in the gene set.

$$Sj = ( {\sum }_{i = 1}^{N} Zi , j ) / \sqrt{N}$$

Sj is the gene set activity score of the given gene set in sample j.

N—number of genes in gene set.

Table 5 provides the gene set activity scores for 3 gene sets across all cell lines.

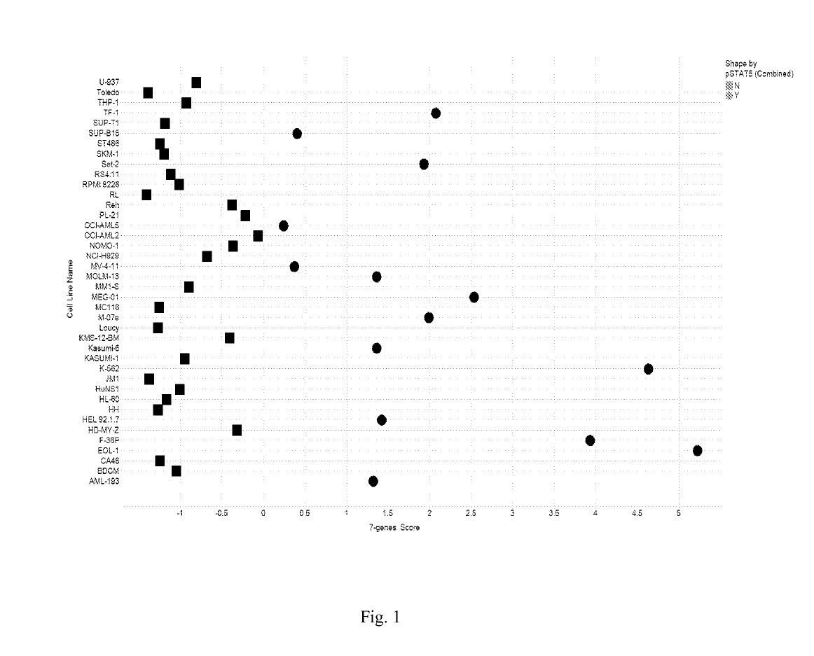

For the 3 gene sets, the probability associated with the Student's t-Test between gene set activity scores for p-STAT5 positive and p-STAT5 negative cell lines was calculated using data from independent validation cell lines set and in all cell lines from enrichment and validation sets combined. Student's t-Test was run using two-tailed distribution and heteroscedastic settings. Table 5 provides the results for 3 gene sets in the validation set cell lines and in all cell lines. As can be seen from Table 6, all 3 gene sets have p-values below 0.05 in the independent validation set. The lowest p-value is observed for 7-gene signature in cell lines set 1 and set 2 combined. FIG. 1 shows relationship between p-STAT5 status and 7-gene signature gene set activity scores across all cell lines. This figure demonstrates the ability of the signature to discriminate between p-STAT5 positive and p-STAT5 negative haematopoietic cell lines.

In summary, we believe that the 3 gene sets listed in Table 4 provide a meaningful way to correlate gene expression levels to STAT5 activation in haematopoietic malignancies. It is technically more feasible and reliable than either immunohistochemistry-based methods or gene signature with much larger gene sets.

TABLE 5

| | | | | | pSTAT5 | |
|---|---|---|---|---|---|---|
| Cell Line | 4-genes | 6-genes | 7-genes | pSTAT5 set 1 | set2* | pSTAT5 |
| Name | Score | Score | Score | (enrichment) | (validation) | Combined |
| THP-1 | −0.82 | −0.88 | −0.93 | N | | N |
| PL-21 | −0.72 | −0.41 | −0.22 | | N | N |
| OCI-AML2 | 0.43 | 0.3 | −0.07 | | N | N |
| NOMO-1 | 0.12 | −0.06 | −0.37 | | N | N |
| HL-60 | −0.83 | −0.92 | −1.17 | | N | N |
| KASUMI-1 | −0.56 | −0.7 | −0.95 | | N | N |
| SKM-1 | −0.88 | −0.94 | −1.2 | | N | N |
| MM1-S | −0.68 | −0.61 | −0.9 | N | | N |
| ST486 | −1.02 | −0.99 | −1.25 | N | | N |
| NCI-H929 | −0.6 | −0.55 | −0.68 | N | | N |
| JM1 | −0.99 | −1.1 | −1.38 | N | | N |
| Loucy | −1.03 | −1 | −1.27 | N | | N |
| RPMI 8226 | −0.71 | −0.73 | −1.02 | N | | N |
| Toledo | −0.98 | −1.11 | −1.39 | N | | N |
| MC116 | −1.06 | −1.01 | −1.26 | N | | N |
| Reh | 0.14 | 0 | −0.38 | N | | N |
| KMS-12-BM | -0.16 | −0.04 | −0.41 | N | | N |
| RS4; 11 | −0.7 | −0.85 | −1.12 | N | | N |
| BDCM | −0.87 | −0.94 | −1.05 | N | | N |
| U-937 | −0.48 | −0.5 | −0.81 | N | | N |
| HD-MY-Z | −0.85 | −0.74 | −0.32 | N | | N |
| HuNS1 | −0.76 | −0.71 | −1.01 | N | | N |
| SUP-T1 | −0.89 | −0.92 | −1.19 | N | | N |
| CA46 | −0.94 | −0.98 | −1.25 | N | | N |
| RL | −1.12 | −1.13 | −1.41 | N | | N |
| HH | −1.01 | −0.98 | −1.27 | N | | N |
| MOLM-13 | 2.13 | 1.79 | 1.36 | Y | | Y |
| AML-193 | 2.46 | 1.74 | 1.32 | Y | | Y |
| Set-2 | 1.72 | 2.38 | 1.93 | Y | | Y |
| TF-1 | 1.65 | 2.63 | 2.07 | Y | | Y |
| HEL 92.1.7 | 1.7 | 1.38 | 1.42 | Y | | Y |
| EOL-1 | 7.46 | 5.98 | 5.22 | | Y | Y |
| F-36P | 4.32 | 4.55 | 3.93 | | Y | Y |
| Kasumi-6 | 2.47 | 1.77 | 1.36 | | Y | Y |
| MV-4-11 | 0.81 | 0.66 | 0.37 | | Y | Y |
| M-07e | 3.06 | 2.34 | 1.99 | | Y | Y |
| OCI-AML5 | 1 | 0.64 | 0.24 | | Y | Y |
| K-562 | 6.12 | 4.92 | 4.63 | Y | | Y |
| SUP-B15 | 1.21 | 0.69 | 0.4 | Y | | Y |
| MEG-01 | 3.09 | 2.94 | 2.53 | Y | | Y |

*only samples unique in set 2 were used for signature validation

TABLE 6

| | 4-genes set | 6-genes set | 7-genes set |
|---|---|---|---|
| Cell lines set | t-test p-value | t-test p-value | t-test p-value |
| Validation | 0.016535 | 0.0171543 | 0.0202114 |
| (set 2) | | | |
| Enrichment + | 1.4144E−05 | 6.443E−06 | 6.273E−06 |
| validation (set 1 | | | |
| and set 2) | | | |

### Example 2

### Use of Gene Signature to Stratify a Patient Population with Activated JAK/STAT5 Signaling for Treatment with JAK/STAT Inhibitor

The STAT5 gene signature was then used to examine pharmacodynamic response to (R)-3-cyclopentyl-3-[4-(7H-pyrrolo[2,3-d]pyrimidin-4-yl)-1H-pyrazol-1-yl]propanenitrile in a preclinical setting. Reagents used are shown in Table 7.

TABLE 7

| Part # | Reagent | Supplier |
|---|---|---|
| 4369510 | Taqman gene expression master mix | ABI/Life Technologies |
| 4331182 | ID1 Hs03676575_s1 (predesigned gene expression assay) | ABI/Life Technologies |
| 4331182 | SOCS2 Hs00919620_m1 (predesigned gene expression assay) | ABI/Life Technologies |
| 4332078 | Custom design: | ABI/Life Technologies |
| | CISH Forward | |
| | Primer: CTGTGCATAGCCAAGACCTTCTC | |
| | Reverse Primer: CGTAATGGAACCCCAATACCA | |
| | Probe: CTTCGGGAATCTGG | |
| 4332078 | Custom design: PIM1 | ABI/Life Technologies |
| | Forward Primer: TGCTCAAGGACACCGTCTACAC | |
| | Reverse Primer: GGATCCACTCTGGAGGGCTAT | |
| | Probe: CTTCGATGGGACCCGAG | |
| 4331182 | housekeeping gene: TBP Hs99999910_m1 | ABI/Life Technologies |
| 4331182 | housekeeping gene: GUSB Hs99999908_m1 | ABI/Life Technologies |

Seven hematologic tumor cell lines (5 positive for pSTAT5 (AML-193, Hel 92.1.7, Set2, TF-1 and UKE-1) and 4 negative for pSTAT5 (RPM18226, U937, Relt and PL-21) were treated with (R)-3-cyclopentyl-3-[4-(7H-pyrrolo[2,3-d]pyrimidin-4-yl)-1H-pyrazol-1-yl]propanenitrilem, 0.2 μM or 1 μM, and samples were collected at 4 hr and 24 hr after treatment. Phospho-STAT5 was examined by western blot analysis, and the expression of the four signature genes was determined by qPCR. The RNA expression level (ΔCt) of each individual gene in the signature was determined by subtracting the average Ct for the signature gene from the average Ct of the two housekeeper genes (GUSB and TBP). For the normalized relative expression levels the DMSO control treatment ΔCt were set to one and all other treatments the gene Ct values are relative to this value.

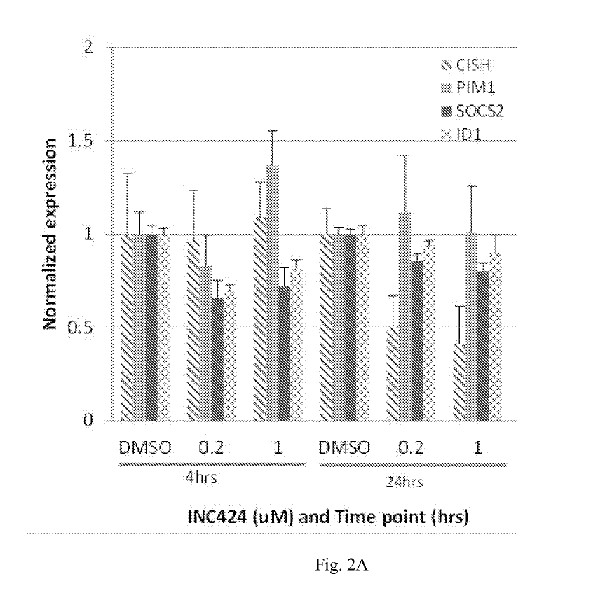

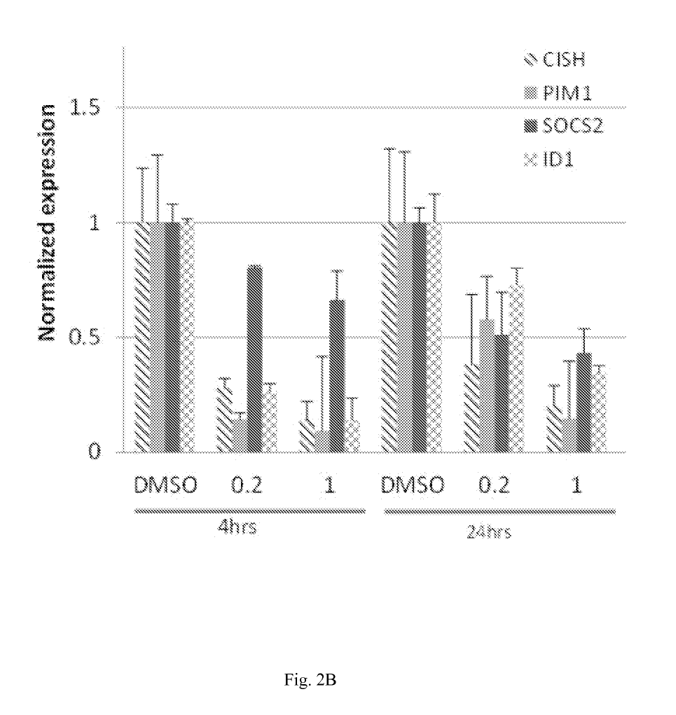

In the pSTAT5 negative cell lines, there was no clear effect by (R)-3-cyclopentyl-3-[4-(7H-pyrrolo[2,3-d]pyrimidin-4-yl)-1H-pyrazol-1-yl]propanenitrile on pSTAT5 modulation or changes in signature gene expression (RPMI 8226 in FIG. 2A). In the pSTAT5 positive cell lines, (R)-3-cyclopentyl-3-[4-(7H-pyrrolo[2,3-d]pyrimidin-4-yl)-1H-pyrazol-1-yl]propanenitrile down-modulated pSTAT5, and there was a corresponding reduction of the expression of the signature genes (TF-1 in FIG. 2B).

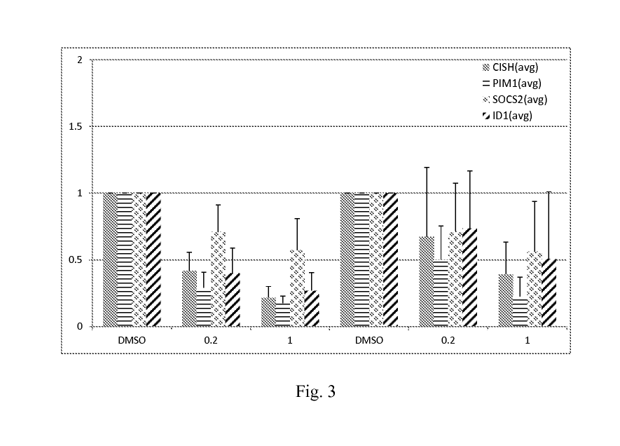

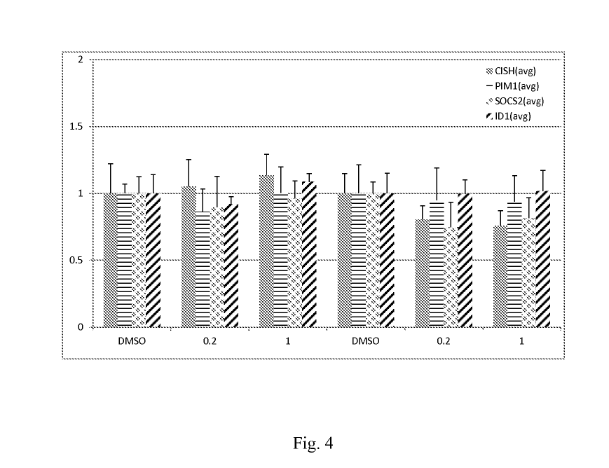

The experiments were performed again with the composite of the modulation of 4 gene signature expression following treatment with (R)-3-cyclopentyl-3-[4-(7H-pyrrolo[2,3-d]pyrimidin-4-yl)-1H-pyrazol-1-yl]propanenitrile across the 5 positive for pSTAT5 (AML-193, Hel 92.1.7, Set2, TF-1 and UKE-1) as shown in FIGS. 3 and 4 negative for pSTAT5 (RPM18226, U937, Relt and PL-21) as shown in FIG. 4.

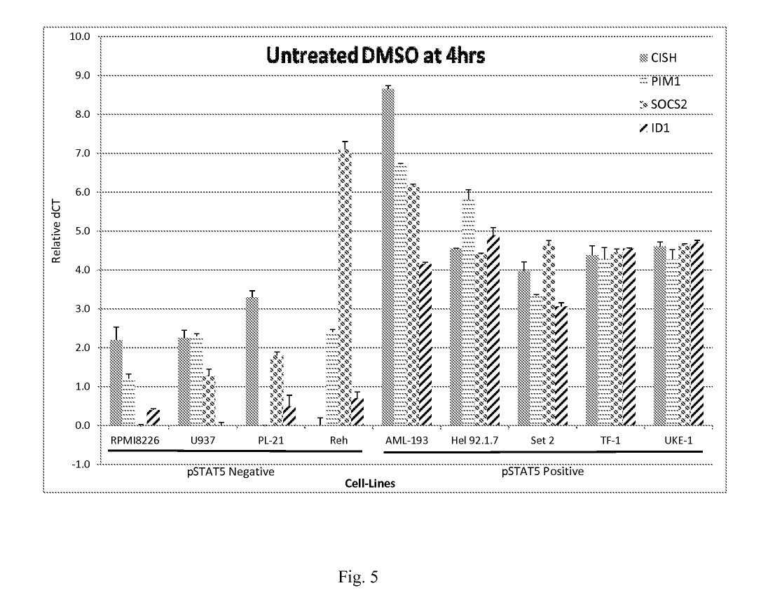

An analysis was also performed on DMSO untreated hematologic tumor cell lines positive for pSTAT5 and negative for pSTAT5 and the RNA expression level (ΔCt) of each individual gene in the signature was determined As shown in FIG. 5 tumor cell lines positive for pSTAT5 had a much higher level of expression of the signature genes.

The results thus prove that the gene signatures described herein can be used to stratify or select for a patient population with activated JAK/STAT5 signaling who could potentially benefit from treatments targeting the JAK/STAT5 signaling pathway. Furthermore, the signature is a consistent predicator of (R)-3-cyclopentyl-3-[4-(7H-pyrrolo[2,3-d]pyrimidin-4-yl)-1H-pyrazol-1-yl]propanenitrile pharmacodynamic effects.

### Example 3

### Tumor Xenograft Study

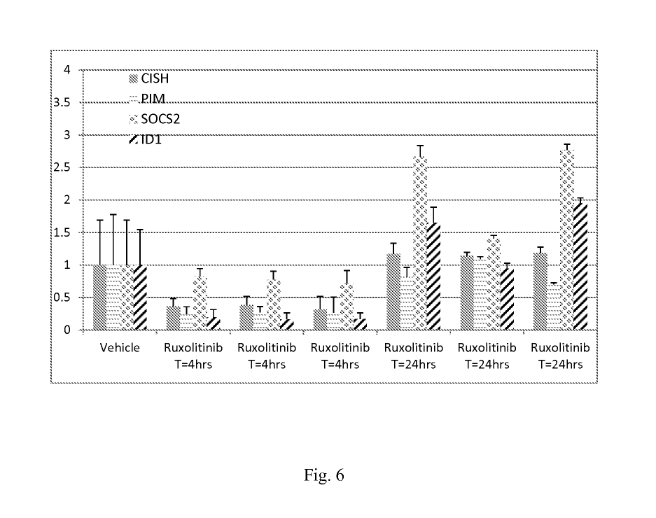

The modulation of gene signature by (R)-3-cyclopentyl-3-[4-(7H-pyrrolo[2,3-d]pyrimidin-4-yl)-1H-pyrazol-1-yl]propanenitrile (ruxolitinib) was further examined in vivo. UKE-1 cells were implanted in female NOD.SCID mice (Harlan) at 1x10e7 cells/mouse. Single dose of (R)-3-cyclopentyl-3-[4-(7H-pyrrolo[2,3-d]pyrimidin-4-yl)-1H-pyrazol-1-yl]propanenitrile was administered P.O. at 60 mg/kg when tumors reached ˜500 mg. Tumor samples were collected at 4 and 24 hours after treatment. The modulation of pSTAT5 in tumor lysate was examined by Western. The modulation of 4-gene signature by (R)-3-cyclopentyl-3-[4-(7H-pyrrolo[2,3-d]pyrimidin-4-yl)-1H-pyrazol-1-yl]propanenitrile in this tumor model is consistent to that observed in vitro (FIG. 6).

### Example 4

### Examination of the Gene Signature in Human Hematological Maligancies

The 4-gene signature was applied to a large collection of gene expression profiles which included about 7,200 human hematological cancer samples. ALL samples including acute lymphoblastic B cell leukemia, acute lymphoblastic leukemia, acute lymphoblastic T cell leukemia, acute myeloid leukemia, acute myeloid leukemia associated with MDS, angioimmunoblastic T cell lymphoma, B cell prolymphoctic leukaemia, chronic myeloid leukemia, juvenile myelomonocytic leukemia, mycosis fungoides sezary syndrome, myelodysplastic syndrome, MDS and precursor T cell lymphoblastic lymphoma have positive signature scores, whereas indications such as T cell lymphoma leukemia, anaplastic large cell lymphoma, B cell lymphoma unspecified, Burkett lymphoma, chronic lymphocytic leukemia and lymphocytic lymphoma, diffuse large B cell lymphoma, follicular lymphoma, hairy cell leukemia, Hodgkin lymphoma, MALT lymphoma, Mantle Cell lymphoma, marginal zone lymphoma, NK T cell lymphoma, peripheral T cell lymphoma unspecified, plasma cell myeloma and T cell lymphoblastic leukaemia exhibit low (negative) signature scores.

1. A method of selecting a subject having a hematological malignancy for treatment with a JAK/STAT inhibitor, the method comprising determining the level of mRNA expression of at least two or more biomarkers listed in Table 1 in a biological sample derived from the subject, thereby to predict an increased likelihood of response to a JAK/STAT inhibitor and selecting a patient likely to respond to a JAK/STAT inhibitor based on the level of mRNA expression compared to a control.

2. The method according to claim 1, comprising determining the level of expression of any three biomarkers in Table 1.

3. The method according to claim 1, comprising determining the level of expression of any four biomarkers in Table 1.

4. The method according to claim 3, wherein the biomarkers comprise PIM1, CISH SOCS2, and ID1.

5. The method according to claim 1, comprising determining the level of expression of any six biomarkers in Table 1.

6. The method of claim 5, wherein the at least six biomarkers comprise PIM1, CISH, SOCS2, ID1, LCN2, and EPOR.

7. The method of claim 1, comprising determining the level of expression of PIM1, CISH, SOCS2, ID1, LCN2, EPOR and EGR1.

8. The method according to claim 1, wherein the JAK/STAT inhibitor is (R)-3-cyclopentyl-3-[4-(7H-pyrrolo[2,3-d]pyrimidin-4-yl)-1H-pyrazol-1-yl]propanenitrile, or a pharmaceutically acceptable salt thereof.

9. The method according to claim 1, wherein the hematological malignancy is leukemia, lymphoma or myeloma.

10. A method of treating a subject having a hematological malignancy with a JAK/STAT inhibitor, the method comprising either
administering a therapeutically effective amount JAK/STAT inhibitor to a selected patient on the basis that the selected patient has been determined to have an increased level of mRNA expression of two or more biomarkers listed in Table 1 compared to a control; or
administering a therapeutically effective amount of an inhibitor which is not a JAK/STAT inhibitor to the selected subject on the basis that the sample does not an increased level of mRNA expression of one or more biomarkers listed in Table 1 compared to a control.

11. The method according to claim 10, wherein the biomarkers comprise PIM1, CISH SOCS2, and ID1.

12. The method of claim 10, wherein the biomarkers comprise PIM1, CISH, SOCS2, ID1, LCN2, and EPOR.

13. The method of claim 10, wherein the biomarkers comprise PIM1, CISH, SOCS2, ID1, LCN2, EPOR and EGR1.

14. A method of treating a subject having a hematological malignancy with a JAK/STAT inhibitor, the method comprising:
determining the level of expression of at least two or more biomarkers listed in Table 1 in a biological sample derived from the subject, and either
administering a therapeutically effective amount JAK/STAT inhibitor to a selected patient on the basis that the selected patient has been determined to have an increased level of mRNA expression of two or more biomarkers listed in Table 1 compared to a control; or
administering a therapeutically effective amount of an inhibitor which is not a JAK/STAT inhibitor to the selected subject on the basis that the sample does not an increased level of mRNA expression of two or more biomarkers listed in Table 1 compared to a control.

15. The method according to claim 14, wherein the expression of the biomarkers determined are PIM1, CISH SOCS2, and ID1.

16. The method of claim 14, wherein biomarkers comprise PIM1, CISH, SOCS2, ID1, LCN2, and EPOR.

17. The method of claim 14, wherein the biomarkers comprise PIM1, CISH, SOCS2, ID1, LCN2, EPOR and EGR1.

18-27. (canceled)

28. The method of claim 10, wherein the JAK/STAT inhibitor is (R)-3-cyclopentyl-3-[4-(7H-pyrrolo[2,3-d]pyrimidin-4-yl)-1H-pyrazol-1-yl]propanenitrile, or a pharmaceutically acceptable salt thereof.

29. The method of claim 14, wherein the JAK/STAT inhibitor is (R)-3-cyclopentyl-3-[4-(7H-pyrrolo[2,3-d]pyrimidin-4-yl)-1H-pyrazol-1-yl]propanenitrile, or a pharmaceutically acceptable salt thereof.


SEQUENCE LISTING (6 sequences)

SEQ ID NO: 1 - DNA, 23 bp, Artificial Sequence
  Oligonucleotide
ctgtgcatagccaagaccttctc

SEQ ID NO: 2 - DNA, 21 bp, Artificial Sequence
  Oligonucleotide
cgtaatggaaccccaatacca

SEQ ID NO: 3 - DNA, 14 bp, Artificial Sequence
  Oligonucleotide
cttcgggaatctgg

SEQ ID NO: 4 - DNA, 22 bp, Artificial Sequence
  Oligonucleotide
tgctcaaggacaccgtctacac

SEQ ID NO: 5 - DNA, 21 bp, Artificial Sequence
  Oligonucleotide
ggatccactctggagggctat

SEQ ID NO: 6 - DNA, 17 bp, Artificial Sequence
  Oligonucleotide
cttcgatgggacccgag